(19) United States
(12) Patent Application Publication
(10) Pub. No.: US 2017/0258571 A1
(43) Pub. Date: Sep. 14, 2017

(54) COMPOSITE COMPRISING FABRIC AND POLYAMPHOLYTE HYDROGEL AND PREPARATION METHOD THEREOF

(71) Applicants: National University Corporation Hokkaido University, Sapporo-shi, Hokkaido (JP); University of Massachusetts, Boston, MA (US)

(72) Inventors: Jian Ping GONG, Sapporo-Shi, Hokkaido (JP); Takayuki KUROKAWA, Sapporo-Shi, Hokkaido (JP); Tao Lin SUN, Sapporo-Shi, Hokkaido (JP); Daniel KING, Boston, MA (US); Alfred CROSBY, Boston, MA (US)

(21) Appl. No.: 15/504,965

(22) PCT Filed: Aug. 19, 2014
(86) PCT No.: PCT/JP2014/072080

§ 371 Date: Feb. 17, 2017

(51) Int. Cl. A61F 2/08; A61L 27/10; A61L 27/16; A61L 27/44; A61L 27/52

(52) U.S. Cl. CPC A61F 2/08; A61L 27/446; A61L 27/52; A61L 27/16; A61L 27/10; A61L 2430/10

(57) ABSTRACT

A composite containing a fabric and a polyampholyte hydrogel is provided. In the composite, the polyampholyte hydrogel is a hydrogel of a polymer containing randomly dispersed cationic and anionic repeat groups and at least a part of the fabric is coated with the polyampholyte hydrogel. A method of preparation of the composite involves steps (a) to (c):
(a) providing a monomer mixture for preparation of a polyampholyte hydrogel;
(b) immersing a fabric in the monomer mixture solution; and
(c) polymerizing monomers in the monomer mixture solution to obtain a precursor of the composite.

### BACKGROUND OF THE INVENTION

Field of the Invention

This invention relates to a composite comprising a fabric and a polyampholyte hydrogel and a method of preparation of the composite.

Discussion of the Background

Anterior Cruciate Ligament (ACL) reconstruction surgeries are one of the most common surgeries performed by orthopedic surgeons. To avoid using autographs or allographs, synthetic materials have been investigated as potential replacements.

Some materials which have been tested are Dacron, Marlex, Teflon, Nylon, and Carbon Fiber.[NP-2] It is important that the materials which are used can support the high load placed upon these ligaments. Carbon Fiber has been especially of interest as a replacement material because of its extremely high stiffness. Initial results with neat carbon fiber replacements were promising.[NP-3,4] However problems were encountered, including fracture, fraying and spreading of carbon in the body, irritation and poor healing of the surrounding area, and accumulation of carbon in regional lymph nodes.[NP-1,2,5]
NP-1: K. Miller, J. E. Hsu, L. J. Soslowsky, in Comprehensive Biomaterials, 2011, pp. 257-279.
NP-2: A. A. Amis, S. A. Kempson, J. R. Campbell, J. H. Miller, Journal of Bone and Joint Surgery 1988, 70-B, 628-634.
NP-3: D. H. R. Jenkins, B. McKibbin, Journal of Bone and Joint Surgery 1980, 62, 497-499.
NP-4: D. H. R. Jenkins, Journal of Bone and Joint Surgery 1978, 60-B, 520-522.
NP-5: S. Ramakrishna, J. Mayer, Composites Science and . . . 2001, 61, 1189-1224.

Some researchers have also attempted to use hydrogels as ligament replacements, but they lack the mechanical properties to support high loads. There is some mention in the patent literature of hydrogel and fabrics as prosthetics. [P-1, 2, 3, 4]
P-1: U.S. Pat. No. 6,629,997.
P-2: U.S. Pat. No. 6,187,043.
P-3: U.S. Pat. No. 6,027,744.
P-4: U.S. Pat. No. 4,919,667.

However the brittleness of traditional hydrogels may have prevented these methods from entering the market.

In addition to the application in ACL reconstruction surgeries, the applications listed below are those in which high toughness is desired, such as skin grafts, flexible tear resistant clothing, bandaging, bullet-proofing, etc.

In bandages, there is very little in the literature about fabric/hydrogel bandages, however there is some mention in the patent literature. In these systems fabric are used as support, and the hydrogel is used for wound closure, as they are generally non-toxic, consist mostly of water, and can promote wound healing. [P-5 to P-8]
P-5: Absorptive wound dressing for wound healing promotion—U.S. Pat. No. 5,695,777;
P-6: Dressing material based on a hydrogel, and a process for its production—U.S. Pat. No. 4,552,138;
P-7: Reinforced, laminated, impregnated, and composite-like materials as crosslinked polyvinyl alcohol hydmgel structures—U.S. Pat. No. 6,855,743:
P-8: Hydrogel Laminate, Bandages, and composites and methods for forming the same—U.S. Pat. No. 5,674,346

However, within these patents there is no obvious mention of utilizing these composite materials to make extremely tough or tear resistant bandages.

Fabric composites have been looked at and utilized before as high strength and toughness materials. Some of these are for ballistics and blast resistance.
P-9: Reinforced film for blast resistance protection—U.S. Pat. No. 8,039,102;
P-10: Impact resistive composite materials and methods for making same—U.S. Pat. No. 7,608,322;
P-11: Body armor with improved knife-stab resistance formed from flexible composites—U.S. Pat. No. 7,288,493;
P-12: Ballistic-resistant article and process for making the same—U.S. Pat. No. 6,268,301;
P-13: Ballistic fabric laminates—U.S. Pat. No. 6,846,758;
P-14: Armor panel—U.S. Pat. No. 5,789,327;
P-15: Method for improving the energy absorption of a high tenacityfabric during a ballistic event—U.S. Pat. No. 5,595,809;
P-16: Lightweight armor and ballistic projectile defense apparatus—U.S. Pat. No. 8,375,839).

There are materials used for airbag technologies in vehicles.
P-17: Silicone composition for coating substrates made of textile material—U.S. Pat. No. 6,586,551;
P-18: Silicone coated textile fabrics—U.S. Pat. No. 6,354,620; etc.

Finally, some patents mention fabrics which exhibit high tear strength.
P-19: Textile-reinforced composites with high tear strength—U.S. Pat. No. 7,549,303;
P-20: Method of making a cut and abrasion resistant laminate—U.S. Pat. No. 6,280,546;
P-21: Elastic composite structure—U.S. Pat. No. 7,153,789;
P-22: Composite elastic material—U.S. Pat. No. 6,706,364;
P-23: Cut resistant yarn, fabric and gloves—U.S. Pat. No. 5,119,512,
P-24: Fiber reinforced thermosetting resin compositions with coated fibers for improved toughness—U.S. Pat. No. 4,737,527;
P-25: Method of manufacture of silicone elastomer coated cloth—U.S. Pat. No. 4,478,895.

However in these examples, none of the composite materials utilize hydrogels, or any type of elastic materials which contain water. In many cases, the matrix material is thermosetting, which would result in the composite lacking any flexibility.

Recently, new polyampholyte (PA) hydrogels produced by Sun et. al. have shown the ability to maintain these features through a macromolecular architecture that also features self-healing, and one-pot synthesis abilities.[NP-6,7]
NP-6: T. L. Sun, T. Kurokawa, S. Kuroda, A. Bin Ihsan, T. Akasaki, K. Sato, M. A. Haque, T. Nakajima, J. P. Gong, Nature materials 2013, 12, 1-6.
NP-7: A. Bin Ihsan, T. L. Sun, S. Kuroda, M. A. Haque, T. Kurokawa, T. Nakajimac, J. P. Gong, J. Mater. Chem. B, 2013, 1, 4555-4562
   Previous work from the Crosby Research Group has shown that embedding soft elastomers with stiff fabrics can result in very interesting composite materials.[NP-8 to NP-10] Double network hydrogels devolved in the Gong Research Group have extremely high toughness, while having moderate ultimate tensile strength and low friction.[NP 11-14].
NP-8: M. D. Bartlett, A. B. Croll, D. R. King, B. M. Paret, D. J. Irshick, A. J. Crosby, Advanced Materials 2012, 24, 1078-1083.
NP-9: S. A. Pendergraph, M. D. Bartlett, K. R. Carter, A. J. Crosby, ACS Applied Materials & Interfaces 2012, 4, 6640-5.
NP-10: D. R. King, M. D. Bartlett, C. A. Gilman, D. J. Irschick, A. J. Crosby, in prep 2013.
NP-11: J. P. Gong, Y. Katsuyama, T. Kurokawa, Y. Osada, Advanced Materials 2003, 15, 1155-1158. Revised 12/17/12 4
NP-12: J. P. Gong, T. Kurokawa, T. Narita, G Kagata, Y. Osada, G Nishimura, M. Kinjo, Journal of the American Chemical Society 2001, 123, 5582-5583.
NP-13: T. Tominaga, N. Takedomi, H. Biederman, H. Furukawa, Y. Osada, J. P. Gong, Soft Matter 2008, 4, 1033.
NP-14: K. Yasuda, J. P. Gong, Y. Katsuyama, A. Nakayama, Y. Tanabe, E. Kondo, M. Ueno, Y. Osada, Biomaterials 2005, 26, 4468-4475.

Utilizing a fabric and PA hydrogel composite could allow for high loads (from the fiber) while the PA hydrogel could prevent wear and rejection from the body. Incorporating these new hydrogels into fabric will allow for new materials with unique properties that are currently lacking in the literatures and patents.

### SUMMARY OF THE INVENTION

A feature of the present invention provides a composite comprising a fabric and a polyampholyte (PA) hydrogel.

A feature of the present invention relates to a method of preparation of the composite.

According to one embodiment of the present invention, it allows us to make tear resistant materials that are extremely flexible, and are made of inexpensive materials. Although there is a large amount of prior work on blast resistant and tear resistant materials, there has not been any prior invention which claims to be extremely tear resistant, yet made of a hydrogel comprising water.

Other exemplary embodiments and advantages of the present invention may be ascertained by reviewing the present disclosure and the accompanying drawings.

### BRIEF DESCRIPTION OF THE DRAWINGS

The present invention will be described in the following text by the exemplary, non-limiting embodiments shown in the figures, wherein:

FIG. 1 is a computer-aided drawing of one embodiment of the composites of the present invention. Left: a side view; right: a projection view.

FIG. 2 is an image of testing setup for the tearing test.

FIG. 3 is a tearing test result showing tear strength at breakage on one embodiment of the composite of the present invention. Comparison of a neat glass fiber fabric, a PA hydrogel, a combination of the fabric and a SN hydrogel, the SN hydrogel.

FIG. 4 shows force vs. displacement curve for 20 mm wide samples of the composite of the present invention, a neat glass fiber fabric, a combination of the fabric and a SN hydrogel.

FIG. 5 shows microscopy images of glass fiber fabric and PA hydrogel between the fibers of the fabric of one embodiment of the composite of the present invention.

FIG. 6 is a microscopy image showing deformation in the tearing region of the fabric (macro scale)

FIG. 7 is a tensile stress-strain measurement result on one embodiment of the composite of the present invention.

FIG. 8 is a tensile stress-strain measurement result on a neat glass fabric.

### DESCRIPTIONS OF THE EMBODIMENTS

The following preferred specific embodiments are, therefore, to be construed as merely illustrative, and not limitative of the remainder of the disclosure in any way whatsoever. In this regard, no attempt is made to show structural details of the present invention in more detail than is necessary for the fundamental understanding of the present invention, the description taken with the drawings making apparent to those skilled in the art how the several forms of the present invention may be embodied in practice.

This invention relates to a composite comprising a fabric and a PA hydrogel which is relatively soft and viscoelastic. The fabric can be a woven fabric or unwoven fabric and the unwoven fabric can be a knitted fabric. A woven fabric is obtained by producing the interlacing of warp fibers and weft fibers in weave styles, such as plain, twill, or satin styles and so on. A woven fabric is preferred because the integrity of the woven fabric is maintained by the mechanical interlocking of the fibers, and their drape, surface smoothness and stability can be controlled by the weave styles based on the design requirements.

The fabric can be made from inorganic material fibers, organic material fibers, biomaterial fibers or a mixture thereof. Inorganic material fibers are composed of inorganic chemical compounds, based on natural elements, which are resistant to high temperatures and high mechanical strength. Examples of the inorganic material fibers include glass fibers, carbon fibers, ceramics fibers and metals fibers. These inorganic materials fibers are commonly applied infields that require high performance materials, such as building materials, protective clothing, parts of airplanes, spaceships, and so on. Organic material fibers are made from synthesized polymers or small molecules, which are polymerized into long linear chains. Examples of the organic material fibers include synthetic polymer fibers such as nylon fibers, aramid fibers, polyalkylene fibers such as polyethylene fibers, polypropylene fibers. These organic material fibers with high mechanical performance and thermal stability are reinforced in the polymer matrix, which are applied in fields such as the automotive industry, railways, aerospace, and so on. Biomaterial fibers are made from plants and animals, which are low cost, low density, high toughness, renewable and biodegradable. Examples of biomaterial fibers include cellulose fibers and cotton fibers. These biomaterials fibers are widely reinforced with a polymer matrix in the textile, and automotive industries. The fabric is preferably comprised of long fibers. Woven and non-woven fabrics that are commercially available can be used. The fabric can be in a form of a sheet, a tape, a strip, a tube, a rod, and so on. The preferred composite is produced by the fabric with hydrophilic surface, such as glass fiber, and PA hydrogel.

In the composite of this invention, at least a part of the surface of the fabric is coated with the PA hydrogel. The PA hydrogel can be coated on either surface of the fabric or both surface of the fabric partially or fully. The PA hydrogel is a hydrogel of a polymer bearing randomly dispersed cationic and anionic repeat groups.

The PA hydrogel is selected from the group consisting of random polymers obtained by random polymerization of a cationic monomer and an anionic monomer. The cationic monomer is selected from the group consisting of monomers having an amino group, preferably a quartemized amine group. Examples of the cationic monomer include the monomer selected from the group consisting of 3-(methacryloylamino)propyltrimethylammonium chloride (MPTC), acryloyloxethyltrimethylammonium chloride (DMAEA-Q), methacrylatoethyl trimethyl ammonium chloride (MTAC) and 4-vinylpyridine chloride (4-VPC).

The anionic monomer is selected from the group consisting of a sulfonate group, a sulfonic acid group, a carboxylate group, a carboxyl acid group, a phosphate group, and a phosphoric acid group. Examples of the anionic monomer include the monomer selected from the group consisting of sodium p-styrenesulfonate (NaSS), anionic 2-acrylamido-2-methylpropanesulfonic acid (AMPS), sodium 4-styrenecarboxylic acid (NaSA), acrylic acid (AA), sodium acrylate, methacrylic acid (MAA), and ethylene glycol 1-methacrylate 2-phosphate.

One example of the PA hydrogels is P(NaSS-co-MPTC) hydrogels, randomly copolymerized from concentrated aqueous solutions of oppositely charged monomers, sodium p-styrenesulfonate (NaSS) and 3-(methacryloylamino)propyltrimethylammonium chloride (MPTC). One example of the P(NaSS-co-MPTC) hydrogels exhibits high strength (tensile fracture stress σb. 1.8 MPa), high extensibility (tensile fracture strain εb=7.4), and high toughness (tearing energies T=4000 J m⁻²). The PA hydrogels are self-recoverable due to the non-covalent ionic bonds. In addition to these excellent mechanical properties, these PA hydrogels have excellent biocompatibility and anti-biofouling properties. [NP-6].

Another example of a PA hydrogels is the PA P(NaSS-co-DMAEA-Q) hydrogels synthesized from sodium p-styrenesulfonate (NaSS) and acryloyloxethyltrimethylammonium chloride (DMAEA-Q). DMAEA-Q is relatively hydrophilic in comparison with the cationic monomer MPTC.

The chemical structures of monomers are shown below:

Cationic monomers: MPTC, DMAEA-Q;

Anionic monomers: NaSS, AMPS.

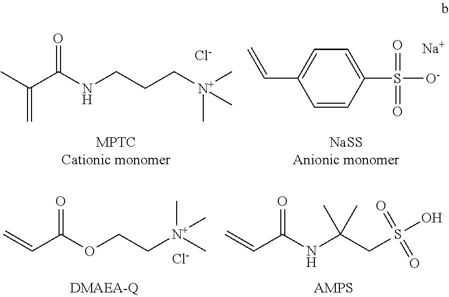

Other examples of PA hydrogels are the P(NaSS-co-MTAC4) and P(NaSS-co-VPC) systems synthesized by using methacrylatoethyl trimethyl ammonium chloride (MTAC) or 4-vinylpyridine chloride (4-VPC) as a cationic monomer. Chemical structures of the cationic monomers for the PA hydrogels are shown below:

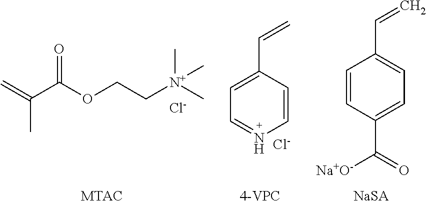

The above PAs are those developed from linear PAs by employing the ionic bond as a reversible sacrificial bond that breaks and reforms dynamically. [NP-6, 7] Owing to the random distribution, the opposite charges on the PAs form multiple ionic bonds of wide distribution in strength. The strong bonds work as permanent cross-linkers, imparting the elasticity of the hydrogel. The weak bonds are fragile and they break under stress, serving as reversible sacrificial bonds. Upon rupture of the weak bonds, the globule polymer chains unfolded, serving as hidden length. These two processes dissipate energy and impart toughness to the hydrogel. The PA hydrogels used in the present invention are supramolecular hydrogels and contain about 50 wt % water, yet are very tough and viscoelastic. An illustration of PA networks with ionic bonds of different strength is shown below. The strong bonds serve as permanent crosslinking points, and the weak bonds act as reversible sacrificial bonds that rupture under deformation.

Neutral PAs form globule structures in aqueous medium by formation of ionic bonds, which leads to phase separation. It is better to determine the optimized charge ratio of the precursor solution to form the neutral PA. The effects of the monomer concentration Cm and the chemical cross-linker density C (CMBAA in case MBAA is used as the chemical cross-linker) on the behavior of the PA is considered when the PA hydrogel is prepared. These two parameters are crucial to control the phase separation of the system. It is preferred that a phase diagram of formulation in the Cm-CMBAA space can be constructed in advance and homogenous hydrogel phase is determined. The mechanical properties of PA hydrogels are dependent on the combinations of the ionic monomer pairs. There is a tendency of that more hydrophilic monomers produced softer and stretchable gels and vice versa.

The polymers are obtained by random copolymerization of a cationic monomer and an anionic monomer and a neutral monomer. The addition of the neutral monomer can adjust the stiffness and toughness of the samples. Examples of the neutral monomer include acrylamide based monomers such as acrylamide (AAm), N-isopropylacrylamide (NIPAM), dimethylacrylamide (DMAAm); vinyl based monomers such as vinyl acetate, vinyl pyridine, styrene; alkylacrylate based monomers such as methylmethacrylate; hydroxyalkyl acrylate based monomers such as hydroxyethylacrylate; and fluorine containing unsaturated monomers such as trifluoroethyl acrylate.

Synthesis of PA Hydrogels and Preparation of the Composite

PA hydrogels are synthesized using a one-step random copolymerization of an anionic monomer and a cationic monomer. Using a one-step random copolymerization, the composite of the present invention can be manufactured in one step.

The present invention further relates to a method of preparation of a composite comprising a fabric and a polyampholyte hydrogel. The method comprises the following steps (a) to (c).

(a) providing a monomer mixture for preparation of a polyampholyte hydrogel;

(b) immersing a fabric in the monomer mixture solution; and

(c) polymerizing monomers in the monomer mixture solution to obtain a precursor of the composite.

(a) Provision of a Monomer Mixture

The monomer mixture can be a mixed aqueous solution of an anionic monomer and a cationic monomer and the aqueous solution is prepared by mixing the monomers with water. The monomer concentration, Cm, influences the behavior of the resulting PA and the preparation of the PA hydrogels. The monomer concentration, Cm, means the total concentration of the anionic monomer and a cationic monomer. The monomer concentration, Cm, is desirably in a range from 1 to 5 mol/L, preferably range from 1.5 to 2.5 mol/L in order to prepare PA hydrogels enabling to provide a tough and stiff, yet flexible composite.

In addition, molar fraction of the anionic monomer F influences the behavior of the resulting PA and the preparation of the PA hydrogels. The molar fraction of the anionic monomer F means a molar fraction of the anionic monomer to the total of the anionic monomer and a cationic monomer. The molar fraction of the anionic monomer F is desirably in a range from 0.4 to 0.6, preferably in a range from 0.5 to 0.54 in order to prepare PA hydrogels that provide tough and stiff, yet flexible composites.

The mixed aqueous solution can further contain a polymerization initiator and/or a cross-linking agent and/or a salt. The polymerization initiator can be ultraviolet initiator such as 2-oxoglutaric acid, sodium 4-[2-(4-morpholino)benzoyl-2-dimethylamino]butylbenzenesulfonate, and phenacylpyridinium oxalate. The concentration of the polymerization initiator can be set in view of the kind of the monomers and the preferable initiator concentration is, but not limited to, in a range from 0.01 mol % to 1 mol % against to total monomer concentration.

The cross-linking agent is preferably a chemical cross-linker having two or more of (meth)acrylate groups in a molecule such as N,N′-methylenebis (acrylamide) (MBAA) and ethyleneglycoldimethacrylate. N,N′-methylenebis (acrylamide) (MBAA) is preferred when an (meth)acrylate based monomer is used. Ethyleneglycoldimethacrylate is preferred when a vinyl monomer such as NaSS and 4-VPC is used. The cross-linking agent can be used in combination or alone. The concentration of the cross-linking agent can vary in view of the monomers and the preferable concentration is, but not limited to, in a range of 0.1 mol % to 10 mol % against to total monomer concentration.

The salt can be an inorganic salt. Examples of the inorganic salt include an alkali metal salt such as LiCl, NaCl, KCl, CsCl, CaCl₂, MgSO₄, K₂SO₄, or MgCl₂. The concentration of the salt can vary in view of the kind of the monomers and the monomer concentration Cm and is not limited specifically but is in a range of 0.1M to 2M, for example. The usage of salt can reduce the viscosity of the mixed aqueous solution and obtain the homogeneous samples. However, the higher the salt concentration, the higher the burden of removal of the salt after polymerization.

(b) Immersion of a Fabric into the Monomer Mixture Solution;

A cell for preparation of the composite is comprised of, but not limited to, a pair of plates such as glass plates facing each other and the thickness between the plates is controlled by a spacer thickness. This spacing is, but not limited to, desirably in a range from 0.001 mm to 10 mm, preferably in a range from 0.01 mm to 5.0 mm, and more preferably in a range from 0.1 mm to 1.0 mm. The plates and the spacer are selected from the material inert with the mixed aqueous solution and the spacer can be a silicone spacer, for example, with a prescribed thickness. The thickness of the spacer decides the thickness of the composite. A fabric is placed in the space made by the plates. This fabric can be woven or knit or unwoven and may range in thickness from 0.001 mm to 1 mm in thickness, and may consist of a plain, twill, or satin weave. The mixed aqueous solution is added into the channel in which the fabric has been placed. The fabric can be placed nominally in the center of the space, or preferentially towards an edge or surface such as 60/40, 70/30, 80/20, etc. distribution of the PA on each side of the fabric. Alternatively, the composite may be prepared by coating the fabric with the mixed aqueous solution on either or both sides of the fabric surface. The coating may be applied by spraying, dipping, knife-over-roll, or other traditional fabric coating methods.

(c) Polymerization of the Monomers to Obtain a Precursor of the Composite

The mixed aqueous solution is cured, for example, by irradiation of ultraviolet light when an ultraviolet initiator is used. Wavelength of the ultraviolet light can be determined in view of the ultraviolet initiator. The irradiation can be conducted in an inert atmosphere, such as argon atmosphere, at an ambient temperature or an increased temperature for 1 minute to 24 hours, preferably from 1 hour to 12 hours, and more preferably from 8 hours to 12 hours, which is adjusted by the power of ultraviolet light. This reaction can take place at an ambient temperature or an increased temperature in an inert atmosphere such as an argon or nitrogen atmosphere. The composite consisted of fabric and PA can be prepared with the thickness from 0.001 mm to 10 mm controlled by the size of spacer.

In this method, the cationic and anionic monomers are essentially copolymerized randomly in the PA hydrogels. The random structure of the copolymer can be confirmed using a ¹H-NMR reaction kinetics study, for example.

After polymerization, the as-prepared gel is immersed in water to reach equilibrium and to wash away the residual chemicals after one week, for example. The amount of the water is not limited specifically and it is preferred to confirm the equilibrium state and disappearance of the residual chemicals in the gel. During this process, the mobile counter-ions of the ionic copolymer as well as the salt used, are removed from the gel, and the oppositely charged ions on the copolymer form stable ionic complexes either through intra- or interchain interactions.

Polymerization conditions and structural features and mechanical properties of various PA hydrogels are shown in the table below.

TABLE 1

| | | Composition | c_{w} | E | | ε_{b} | W_{b} | | R | Tₛ |
|---|---|---|---|---|---|---|---|---|---|---|
| | Hydrogels | Cₘ-f* | (wt %) | (MPa) | σ_{b} (MPa) | (m/m) | (MJ/m²) | tanδ | (%) | (° C.) |
| Polyampholyte | P(NaSS-co-MPTC)⁽¹⁾ | 2.1-0.52 | 54.3 | 2.05 | 1.82 | 7.42 | 7.12 | 0.24 | 76.6 | 48.2 |
| hydrogels | P(NaSS-co-DMAEA-Q)⁽²⁾ | 2.0-0.52 | 52.6 | 0.10 | 0.14 | 15.5 | 1.31 | 0.58 | 95.5 | 17.3- |
| | P(NaSS-co-4-VPC)⁽³⁾ | 2.0-0.5 | 61.1 | 7.92 | 1.25 | 6.42 | 5.05 | 0.24 | 77.1 | 54.2 |
| | P(NaSS-co-MTAC) | 2.0-0.5 | 52.3 | 0.042 | 0.33 | 13.77 | 2.14 | 0.56 | 87.9 | 47.1 |
| Common hydrogels | PAAm | 2-4⁽⁴⁾ | 84.1 | 0.092 | 0.067 | 0.65 | 0.024 | 0.044 | — | — |
| | Single network | | | | | | | | | |
| | PAMPS/PAAm | 1-4/2-0.01⁽⁵⁾ | 86.0 | 0.12 | 1.04 | 12.38 | 8.94 | 0.02 | 61.57 | — |
| | double network | | | | | | | | | |

*Cm and f represent the total ionic monomers concentration (mol/L) and the charge fraction of the anionic monomer, respectively, in the precursor solution used to synthesize the gels. The parameters cw, E, σb, εb. Wb, tanδ, and R are the water content, Young's modulus, fracture stress, fracture strain, work of extension at fracture, loss factor (10 Hz and strain 0.5%), and shock-absorbing ratio, respectively, at room temperature. Ts is the softening temperature determined by the peak of loss factor of the gels.

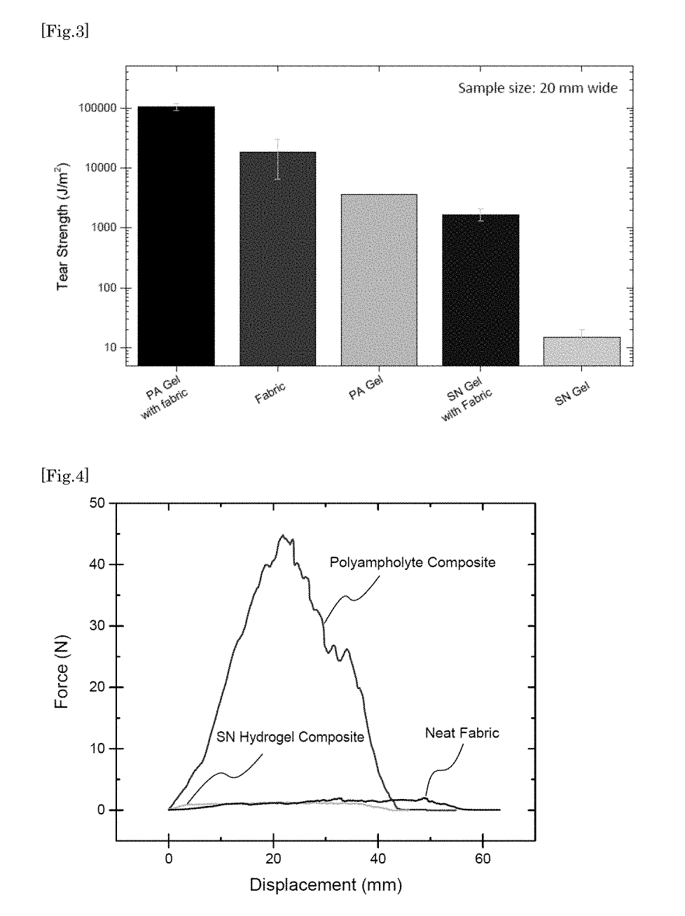

A computer-aided drawing of one embodiment of the composites of the present invention is shown in FIG. 1. Left side is a side view of the composite and right side is a projection view. One embodiment of the composites of the present invention exhibits toughness two orders of magnitude higher than traditional hydrogels (SN gel) (Gc 4800 J/m² vs 10 J/m²). In addition, one embodiment of the composites of present invention exhibits dramatic increase in toughness. For the case of a 20 mm wide strip of the composite comprised of woven glass fiber and PA gel, a Gc value (105,000 J/m²) can be obtained which is an additional two orders of magnitude greater than the neat PA gel, and 4 orders of magnitude higher than a traditional SN gel. (See FIG. 3).

Interestingly, unlike most composites where combining two materials results in an averaging of their mechanical properties, in this case the tear strength is much greater than either materials when tested independently. Also when the control SN gel and fabric was tested, a noticeable decrease in tear strength was observed, which reinforces that this special combination of PA gel and fabric is necessary to see a great increase in tear strength (See the following Examples).

The composite of the present invention can be employed for medical treatment for example for ligament reconstruction surgeries such as Anterior Cruciate Ligament (ACL) reconstruction surgeries. The composite of the present invention can also be employed for bandages, skin grafts, flexible tear resistant clothing or bullet-proofing.

There are many ways this invention improves over the prior art. When used as internal prostheses, fabrics have many issues with regards to tearing, failing, and degrading. Often to prevent this, these stiff substrates would be coated in elastomers. However elastomers often result in a bodily response which can lead to infection or rejection. The coating material, the PA hydrogel, used here contains primarily of water and is non-toxic, and resists cell adhesion.

The composite of the present invention has various utilities. Wound care would benefit from this invention, because the PA hydrogels could create extremely resilient and tear resistant bandages. In the composite of the present invention, the fabric is used as support and the PA hydrogel is used for wound closure, as they are generally non-toxic, consist mostly of water, and can promote wound healing.

### EXAMPLES

The present invention will be described in detail below based on examples. However, the present invention is not limited to the examples.

### 1. Materials

Sodium p-styrenesulfonate (NaSS), Methyl chloride quartemized N, N-dimethylamino ethylacrylate (DMAEA-Q), Acrylamide (AAm), 2-oxoglutaric acid and N,N′-methylenebis (acrylamide) (MBAA) were purchased from the Wako company (Japan). Fabric (S-Glass 4-Harness Satin Weave) (4HS) were purchased from the ACP composite company. In the 4HS, the fill yarn passes over three warp yarns and under one. All of the chemicals were used as purchased without further purification.

### 2. Methods

### 2-1. Synthesis of Materials

Polyampholyte hydrogels (PA) were synthesized using the one-step random copolymerization of an anionic monomer NaSS and a cationic monomer DMAEA-Q. A mixed aqueous solution with the prescribed monomer concentration (Cm=2.0 mol/L) and molar fraction of the anionic monomer (F=0.52), 0.1 mol % ultraviolet initiator, 2-oxoglutaric acid, 0.1 mol % chemical crosslinker MBAA (in a concentration relative to the total monomer concentration), and 0.5M NaCl was poured into a reaction cell. The cell was consisted of a pair of glass plates with 10 mm (L)×10 mm (w)×1 mm(d), where the thickness was controlled by the silicone spacer thickness. The reacted system was irradiated with 365-nm ultraviolet light for 8 h in the atmosphere of argon.

Polyacrylamide hydrogels (PAAm) were produced from the polymerization of 2 mol/L acrylamide monomer, 0.1 mol % 2-oxoglutaric acid and 1.0 mol % chemical crosslinker MBAA (relative to the total monomer concentration) under the UV light.

PA gel/fabric composite were produced by the similar described polymerization process. The fabric 4HS with approximately 0.3 mm thickness were fixed to the center of reaction cell prior to injecting the reaction solution. To produce the PA gel/fabric composite, the polymerization was carried out after filling the PA solution with the same composition to the reaction cell.

Similar to the PA gel/fabric composite production, the PAAm/fabric composite was synthesized by the AAm solution and fabric 4HS.

After polymerization, the as-prepared gel was immersed in a large amount of water for 1 week to allow the gel to reach equilibrium and to wash away the residual chemicals and counter-ions.

### 2-2. Characterization

### Tearing Test:

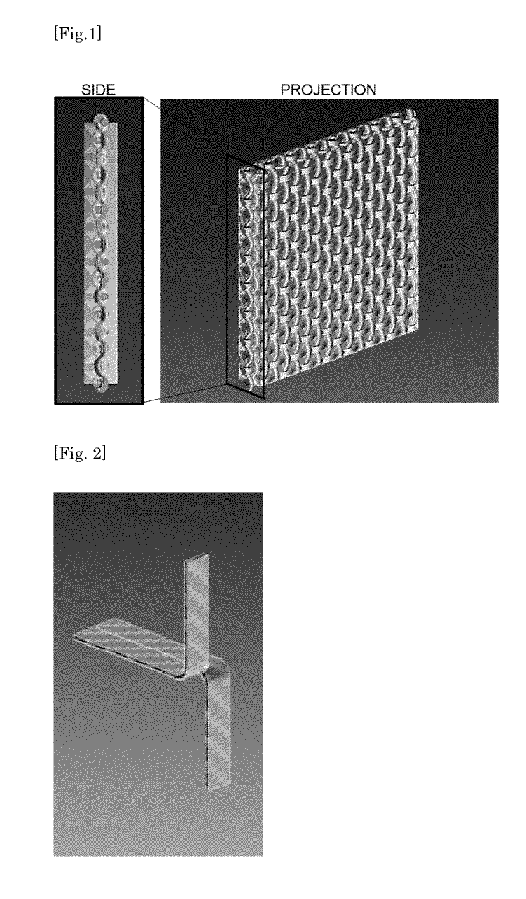

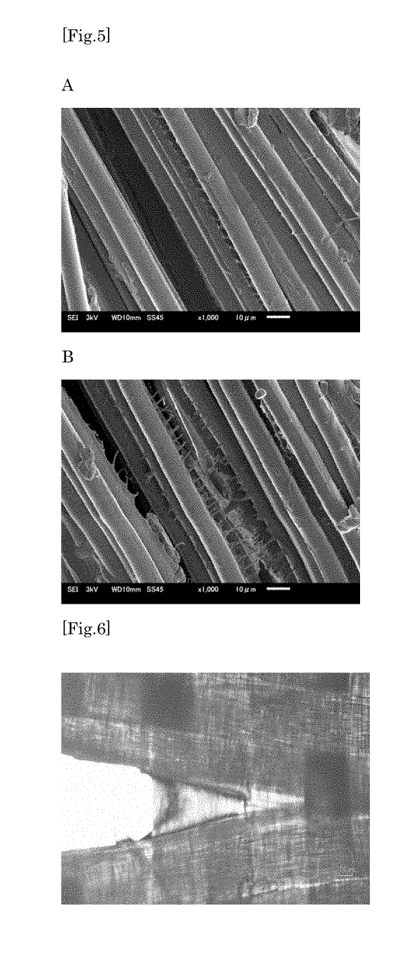

The tearing test was performed to characterize the toughness in air using a commercial test machine (Instron Cat. No 2752-005). The image of testing setup for the tearing test is shown in FIG. 2. The test samples were cut to rectangular shape with 40 mm (L)×20-100 mm (w)×0.5-1.3 mm (t). A cut was made 20 mm along the length, at the center of the width of the sample, to create two arms. The two arms of a test piece were clamped, and then the upper arm was pulled upward at constant velocity 50 mm/min while the tearing force F was recorded. The tearing energy T was calculated at a constant tearing force F using the relation T=2F/w, where w is the thickness of the sample. The results are shown in FIGS. 3 and 4. FIG. 3 shows tear strength at breakage and FIG. 4 shows force vs. displacement curve for 20 mm wide samples of the composite of the present invention, a neat glass fiber fabric, a combination of the fabric and a SN hydrogel. FIG. 5 shows microscopy images of glass fiber fabric and PA hydrogel between the fibers of the fabric of the sample, A is an image of fibrillation in the matrix gel and B is an image of fracture of the fibrils, which is hypothesized to dissipate energy. FIG. 6 is a microscopy image showing deformation in the tearing region of the fabric (macro scale).

### Tensile Test:

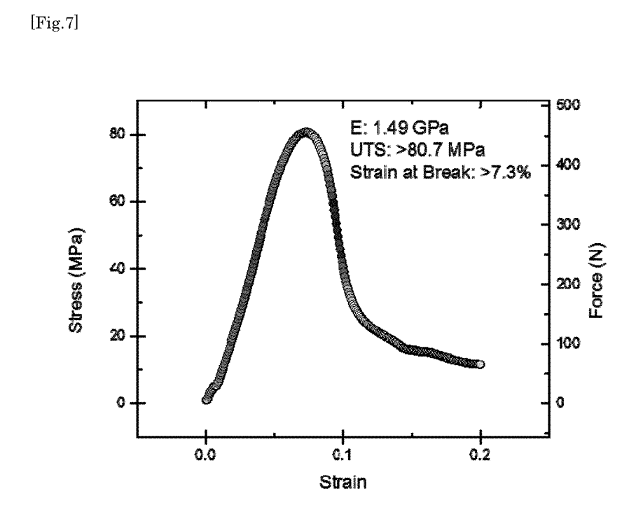

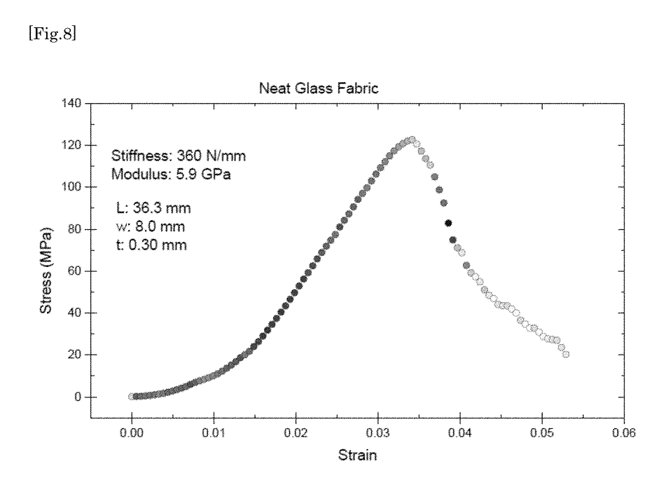

The tensile stress-strain measurements were performed using a tensile-compressive tester (Tensilon RTC-1310A, Orientec Co.) at a deformation rate of 10 mm/min in air. The test was carried out on samples with rectangle shape with the size (25 mm (L)×20 mm (w)×0.85 mm (d). In order to prevent the slipping of the sample at the end of the clamp during the tensile test, polyethylene terephthalate (PET) plates with 2 mm (L)×2 mm (w)×3 mm (d) adhere the ends of samples was used by super glue cyanoacrylate. The PET plates were gripped by the clamp during the tensile test. The results are shown in FIGS. 7 (composite) and 8 (neat glass fabric). The strength of the materials exceeded the strength of the mounting grips, and the ultimate properties of these materials exceed the results presented.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

1. A composite comprising a fabric and a polyampholyte hydrogel, wherein
the polyampholyte hydrogel is a hydrogel of a polymer bearing randomly dispersed cationic and anionic repeat groups; and
at least a part of the fabric is coated with the polyampholyte hydrogel.

2. The composite according to claim 1, wherein the polyampholyte hydrogel is selected from the group consisting of random polymers obtained by random polymerization of at least one cationic monomer and at least one anionic monomer.

3. The composite according to claim 1, wherein the cationic monomer is selected from the group consisting of monomers having at least one quarternized amine group.

4. The composite according to claim 1, wherein the cationic monomer is selected from the group consisting of MTPC, DMAEA-Q, MTAC and 4-VPC.

5. The composite according to claim 1, wherein the anionic monomer is selected from the group consisting of monomers having at least one sulfonate group and monomers having at least one sulfonic acid group.

6. The composite according to claim 1, wherein the anionic monomer is selected from the group consisting of NaSS and AMPS.

7. The composite according to claim 1, wherein the random polymers obtained by random polymerization of a cationic monomer and an anionic monomer and at least one of monomers other than the cationic and anionic monomers.

8. The composite according to claim 1, wherein the monomers other than the cationic and anionic monomers is a monomer comprising at least polymerizable moiety.

9. The composite according to claim 1, wherein the fabric is a woven fabric or unwoven fabric.

10. The composite according to claim 1, wherein the fabric is in a form of a sheet, a tape, or a strip.

11. The composite according to claim 1, wherein the fabric is made of inorganic material, organic polymer material and a mixture thereof.

12. The composite according to claim 1, wherein the fabric is fully coated with the polyampholyte in a thickness of from 0.001 mm to 10 mm.

13. A method of preparation of a composite comprising a fabric and a polyampholyte hydrogel, comprising
(a) providing a monomer mixture for preparation of a polyampholyte hydrogel;
(b) immersing a fabric in the monomer mixture solution; and
(c) polymerizing monomers in the monomer mixture solution to obtain a precursor of the composite.

14. The method according to claim 13, wherein the monomer mixture solution has a monomer content of from 1 mol/L or more.

15. The method according to claim 13, wherein the monomer mixture solution further comprises a polymerization initiator and/or a cross-linking agent.

16. The method according to claim 13, wherein the monomer mixture solution further comprises a salt.

17. The method according to claim 13, wherein the method further comprises a step (d) in which the precursor of the composite is desalted to obtain the composite.

18. The method according to claim 13, wherein the fabric has a hydrophobic surface.

19. The composite of claim 1, wherein the composite is employed for medical treatment.

20. The composite of claim 1, wherein the composite is employed for ligament reconstruction surgeries.

21. The composite of claim 1, wherein the composite is employed for Anterior Cruciate Ligament (ACL) reconstruction surgeries.

22. The composite of claim 1, wherein the composite is employed for bandages, skin grafts, flexible tear resistant clothing or bullet-proofing.
